(19) United States
(12) Patent Application Publication
(10) Pub. No.: US 2026/0151400 A1
(43) Pub. Date: Jun. 4, 2026

(54) USE OF ANTAGONISTS OF NEUROKININ-1 RECEPTOR AND NUCLEOSIDE ANALOGS FOR THE TREATMENT OF HERPESVIRIDAE

(71) Applicant: THE REGENTS OF THE UNIVERSITY OF COLORADO, a body corporate, Denver, CO (US)

(72) Inventors: Maria A. NAGEL, Aurora, CO (US); Andrew N. BUBAK, Aurora, CO (US)

(21) Appl. No.: 19/122,549

(22) PCT Filed: Oct. 19, 2023
(86) PCT No.: PCT/US23/77316

§ 371 Date: Apr. 18, 2025

Related U.S. Application Data
(60) Provisional application No. 63/417,757, filed on Oct. 20, 2022.

(51) Int. Cl. A61K 31/5377; A61K 31/438; A61K 31/522; A61P 31/22

(52) U.S. Cl. CPC A61K 31/5377; A61K 31/438; A61K 31/522; A61P 31/22

(57) ABSTRACT

Methods for decreasing or inhibiting herpesviridae infection, or associated condition, disease, or pathogenesis or other adverse effect of herpesviridae infection including reactivation of herpesviridae in a subject by administering to the subject a synergistic combination of Neurokinin-1 Receptor antagonist and a nucleoside analog.

### CROSS REFERENCE TO RELATED APPLICATION

This application claims the benefit of U.S. Provisional Patent Application Ser. No. 63/417,757, filed Oct. 20, 2022, which is incorporated herein by reference.

### TECHNICAL FIELD

This invention relates generally to treatment of herpesviridae infection by administering Neurokinin-1 Receptor antagonists and a nucleoside antagonist.

### BACKGROUND

Herpesviruses represent a family of DNA viruses that include herpes simplex virus (HSV) 1 and 2, varicella zoster virus (VZV), Epstein-Barr virus (EBV), cytomegalovirus (CMV), human herpesvirus (HHV) 6 and 7 and Kaposi sarcoma-associated herpesvirus (KSHV; or HHV-8). Globally, the World Health Organization estimates that 500 million people are infected with HSV-2 while two-thirds of the population are infected with HSV-1. These viruses cause human diseases ranging from oral cold sores to genital lesions to serious eye conditions that can lead to blindness. In infants, HSV can cause neurological and developmental problems. People infected with HSV also have an enhanced risk of acquiring or transmitting human immunodeficiency virus (HIV).

These viruses have several common exclusive characteristics: primary infections usually occur during infancy or childhood; all herpesviruses become latent following primary infection, residing in the host nervous or hematopoietic systems. Latency is identified by serum antibodies and the antibody positivity rate among adults (seroprevalence) of 65% for HSV 1 and 54% (by age 40) for CMV in the United States and reflects the acquisition rates of these viruses in childhood. During latency, reactivations are common and mostly asymptomatic but result in virus shedding in mucus membranes, which is the source of infection for contacts of asymptomatic shedders. Infections primarily acquired in adulthood tend to be more severe. Symptomatic reactivations are rare among immunocompetent people except for VZV reactivations (zoster or shingles) but are very common among immunocompromised patients.

Varicella zoster virus (VZV) is an exclusively human neurotropic virus that produces varicella on primary infection, after which it establishes latency in ganglionic neurons within cranial nerve, dorsal root, sympathetic, parasympathetic and enteric ganglia along the entire neuraxis, as well as in adrenal glands. With age or immunosuppression, VZV reactivates from one or more ganglia and typically travels peripherally to skin to produce zoster in the corresponding dermatome. However, virus can also travel centrally along nerve fibers to infect the spinal cord and produce myelitis with or without zoster rash.

VZV myelitis is one of several neurological complications of VZV reactivation and presents as a self-limiting paraparesis with or without sensory features and sphincter problems. In immunocompromised individuals, VZV myelopathy can be progressive and fatal with frank virus infection of the spinal cord. Pathogenic mechanisms of VZV myelitis are not well-characterized, mainly because VZV is an exclusively human virus and there are no animal models to study neurological complications of VZV infection. However, postmortem analyses of patients with VZV myelopathy revealed that astrocytes, as well as oligodendrocytes and neurons, in the spinal cord were infected horizontally and longitudinally. The site of cord involvement was associated with VZV reactivation and zoster rash, supporting the notion that after VZV reactivation from dorsal root ganglionic neurons, virus travels peripherally to skin to produce zoster and centrally to the spinal cord to produce myelitis along the same dermatome(s). Additional support that VZV myelitis can be caused by direct VZV invasion of the spinal cord is provided by cases of acute and rapidly progressive, virologically verified VZV myelitis in patients with acquired immunodeficiency syndrome, who improved with aggressive antiviral therapy, as well as cases of acute VZV myelitis in multiple sclerosis patients on immunomodulatory therapy, who also improved upon antiviral therapy.

Of the multiple cell types within the spinal cord, astrocytes are most likely a key contributor to the central nervous system (CNS) spread of infection and the development of VZV myelitis, because astrocytes are the most abundant glial cell type in the CNS with a high capacity for migration and are crucial regulators of neuroinflammation. Current knowledge of VZV infection of astrocytes is limited to brain astrocytes. Postmortem immunohistochemical studies of brain from VZV encephalitis cases showed that brain astrocytes are infected with VZV and are preferentially infected over other cell types. In vitro studies showed that astrocytes isolated from human brain are permissive to VZV infection, with VZV downregulating expression of glial fibrillary protein in these cells.

A major challenge in treating herpesvirus infections is the limited antiviral therapies available that can produce side effects including nephrotoxicity, limited its use for infected patients with kidney disease; in addition, antiviral resistance can develop. For VZV, HSV-1 and HSV-2 infection, there is only 1 antiviral drug class available (DNA synthesis inhibitors). The most commonly used is acyclovir, a prodrug that is cleaved to acyclovir monophosphate (acido-GTP) by viral thymidine kinase within the cell that contains replicating virus. The acyclovir monophosphate is a nucleoside analog that incorporates into viral DNA, resulting in chain termination. The acyclovir monophosphate is also converted by host cell kinases to acyclovir triphosphate, a potent inhibitor of viral DNA replication through competitive inhibition and inactivation of viral DNA polymerase.

Common side effects for oral and intravenous (IV) acyclovir include nausea, vomiting, diarrhea, and headaches; encephalopathy and injection site reactions can be seen with IV administration. Infrequent side effects include agitation, vertigo, confusion, dizziness, oedema, arthralgia, sore throat, constipation, abdominal pain, hair loss, rash and weakness. Rare adverse effects include coma, seizures, neutropenia, leukopenia, crystalluria, anorexia, fatigue, hepatitis, Stevens-Johnson syndrome, toxic epidermal necrolysis, thrombotic thrombocytopenia purpura and anaphylaxis. In 5-10% of patients, IV acyclovir may cause reversible nephrotoxicity because of precipitation of aciclovir crystals in the kidney. The side effect of nephrotoxicity limits acyclovir's use in patients with kidney disease.

Other viral DNA synthesis inhibitors include valacyclovir, brivudin, cidofovir penciclovir, famciclovir, ganciclovir, valganciclovir, idoxuridine, trifluridine and vidarabine. These agents possess variable activity for different herpesviruses notably differing in their anti-CMV activity.

Resistance to acyclovir and nucleoside analogs is rare but can occur in 10-30% of individuals with immunodeficiencies on chronic antiviral prophylaxis (transplant recipients, AIDS individuals). Resistance can occur with deficient or mutated viral thymidine kinase and mutations in the viral DNA polymerase. For individuals who develop resistance and/or have kidney disease, there are no safe alternatives for treatment of herpesvirus infection.

Thus, there is an urgent need for additional effective agents for herpesviridae with better toxicity and resistance profiles than currently available therapies.

### SUMMARY

The inventors have surprisingly discovered that neurokinin-1 receptor (NK-1R) antagonists act synergistically with nucleoside analogs to effectively treat herpesviridae infection and prevent reactivation of latent herpesviridae. This disclosure features methods of treating herpesviridae infection in a subject in need thereof by administering to the human subject a synergistic combination of a Neurokinin-1 Receptor (NK-1R) antagonist selected from the group consisting of aprepitiant, rolapitant, fosaprepitant, lanepitant, befetupitant, pharmaceutically acceptable salts thereof, and combinations thereof, and a nucleoside analog selected from the group consisting of acyclovir, famciclovir, valaciclovir, brivudin, cidofovir, penciclovir, famciclovir, ganciclovir, valganciclovir, idoxuridine, trifluridine, vidarabine, pharmaceutically acceptable salts thereof, and combinations thereof.

In these methods, the NK-1R antagonist and the nucleoside analog may be administered sequentially, concomitantly, or concurrently. The NK-1R antagonist and the nucleoside analog may be administered as a combined formulation.

In these methods, the NK-1R antagonist may be aprepitant or rolapitant or fosaprepitant. The nucleoside analog may be acyclovir or famciclovir or valaciclovir.

In these methods, the NK-1R antagonist may be administered orally or intravenously. The nucleoside analog may be administered orally or intravenously or topically.

In these methods, the subject may have a herpesviridae infection that is an alpha-herpesvirus or a beta-herpesvirus or a gamma-herpesvirus or a combination of these herpes viruses. The subject may have an alpha-herpesvirus infection that is herpes simplex type 1 (HSV-1), herpes simplex type 2 (HSV-2), varicella-zoster virus (VZV), or a combination of these herpes viruses. In these methods, the subject may have a herpesviridae infection that is Epstein-Bar virus (EBV) infection.

In these methods, the herpesviridae in the subject may be in an active state or in a latent state or in a reactivated state.

In these methods, the administration of the synergistic combination of the NK-1R antagonist and the nucleoside analog may reduce, decrease, inhibit, or ameliorate the onset, severity, duration, progression, or frequency of one or more symptoms or pathologies associated with or caused by herpesviridae infection or pathogenesis, or reactivation of herpesviridae from latency, in the subject. The symptoms or pathologies of herpesviridae infection that may be treated include myelitis, myelopathy, neuropathy, pancreatitis, VZV-associated diabetic complications, vasculopathy, lesions, ulcers, canker sore, close sore, rash, boils, gingivostomatitis, herpes gladiatorum, eczema herpeticum, swollen lymph nodes, pneumonitis, pneumonia, hepatitis, meningitis, encephalitis, keratitis, genital herpes, esophagitis, hemiparesis, shingles, chicken pox, mononucleosis, chronic or acute pelvic inflammatory disease (PID), proctitis, colitis, and/or nerve damage. For example, when the herpesviridae infection is in spinal astrocytes in the subject, the pathogenesis that may be treated is VZV myelitis and/or VZV myelopathy. Alternatively, when the herpesviridae infection is in perineurial cells in the subject, the pathogenesis that may be treated is VZV neuropathy. When the herpesviridae infection is in pancreatic islet cells in the subject, the pathogenesis treated may be VZV pancreatitis and/or VZV-associated diabetic complications. When the herpesviridae infection is in keratocytes in the subject, the pathogenesis treated may be VZV keratitis. When the herpesviridae infection is in brain vascular adventitial fibroblasts in the subject, the pathogenesis treated may be VZV vasculopathy.

In these methods, the administration of the synergistic combination of the NK-1R antagonist and the nucleoside analog may stabilize the herpesviridae infection or one or more symptoms or pathologies associated with the herpesviridae infection or pathogenesis or reactivation of herpesviridae from latency.

In these methods, the administration of the synergistic combination of the NK-1R antagonist and the nucleoside analog may reduce herpesviridae titer, viral load, viral replication, viral proliferation or viral protein production, or inhibit increases in herpesviridae titer, viral load, viral replication, viral proliferation, or viral protein production.

In these methods, the subject is typically a human subject. The subject may be immunocompromised, or a candidate for, or has received, an immunosuppressant treatment, or a candidate for, or has received, a tissue or organ transplant.

Another aspect of this disclosure provides kits for administering a synergistic combination of the NK-1R antagonist and the nucleoside analog. These kits may include a pharmaceutical composition comprising a NK-1R antagonist selected from the group consisting of aprepitiant, rolapitant, fosaprepitant, lanepitant, befetupitant, pharmaceutically acceptable salts thereof, and combinations thereof, and a pharmaceutical composition comprising a nucleoside analog selected from the group consisting of acyclovir, famciclovir, valaciclovir, brivudin, cidofovir, penciclovir, famciclovir, ganciclovir, valganciclovir, idoxuridine, trifluridine, vidarabine, pharmaceutically acceptable salts thereof, and combinations thereof, and a package insert. A closely related aspect of this disclosure provides a kit that includes a package insert and a pharmaceutical composition comprising a NK-1R antagonist selected from the group consisting of aprepitiant, rolapitant, fosaprepitant, lanepitant, befetupitant, pharmaceutically acceptable salts thereof, and combinations thereof, and a nucleoside analog selected from the group consisting of acyclovir, famciclovir, valaciclovir, brivudin, cidofovir, penciclovir, famciclovir, ganciclovir, valganciclovir, idoxuridine, trifluridine, vidarabine, pharmaceutically acceptable salts thereof, and combinations thereof.

This Summary is neither intended nor should it be construed as representative of the full extent and scope of the present disclosure. Moreover, references made herein to “the present disclosure,” or aspects thereof, should be understood to mean certain embodiments of the present invention and should not necessarily be construed as limiting all embodiments to a particular description. The present invention is set forth in various levels of detail in this Summary as well as in the attached drawings and the Detailed Description and no limitation as to the scope of the present invention is intended by either the inclusion or non-inclusion of elements, components, etc. in this Summary. Additional aspects of the present invention will become more readily apparent from the Detailed Description, particularly when taken together with the figures.

### BRIEF DESCRIPTION OF FIGURES

FIG. 1A shows the average VZV DNA copy numbers in all treatment groups when primary human spinal astrocytes were infected with VZV and treated 18 hours later with various doses of aprepitant, acyclovir, or a combination of the two drugs.

FIG. 1B shows individual statistical comparisons for select treatment combinations of the treatment groups from FIG. 1A.

FIG. 1C shows the results of BLISS mathematical modeling for drug synergy for select treatment combinations of the treatment groups from FIG. 1A.

FIG. 1D shows the results of ZIP mathematical modeling for drug synergy for select treatment combinations of the treatment groups from FIG. 1A.

### DETAILED DESCRIPTION

The present invention is based, in part, on the unexpected discovery that, while nucleoside analogs, such as acyclovir, are effective as antiviral drugs against Herpesviridae infection, combining the inhibition of herpesviridae by nucleoside analogs with the inhibition of herpesviridae by NK-1 receptor antagonists can more effectively and surprisingly synergistically-treat Herpesviridae infection.

Consequently, this disclosure relates to methods of treating infection or reactivation of herpesviridae in a subject by administering to the subject a synergistic combination of (i) a Neurokinin-1 Receptor (NK-1R) antagonist selected from the group consisting of aprepitiant, rolapitant, fosaprepitant, and pharmaceutically acceptable salts thereof, and (ii) a nucleoside analog selected from the group consisting of acyclovir, famciclovir, valaciclovir, and pharmaceutically acceptable salts thereof.

### Definitions

Unless defined otherwise, technical and scientific terms used herein have the same meaning as commonly understood by one of ordinary skill in the art to which this invention belongs.

The words “comprise,” “comprising,” “include,” “including,” and “includes” when used in this specification and claims are intended to specify the presence of stated features, integers, components, or steps, but they do not preclude the presence or addition of one or more other features, integers, components, steps, or groups thereof.

The terms “treat” and “treatment” refer to both therapeutic treatment and prophylactic or preventative administration, wherein the object is to prevent or slow down (lessen) an undesired physiological change or disorder, as a consequence of the onset, progression, or transmission of a viral infection. For purposes of this invention, beneficial or desired clinical results include, but are not limited to, alleviation of symptoms, diminishment of extent of infection, stabilized (i.e., not worsening) state of infection, delay or slowing of infection progression, amelioration or palliation of the infected state, and reoccurrence (whether partial or total), whether detectable or undetectable. “Treatment” can also mean prolonging survival as compared to expected survival if not receiving treatment. Those in need of treatment include those already with the condition or related disorders as well as those prone to have the condition or disorder or those in which the condition or disorder is to be prevented. In the context of HV infection, prevention specifically includes eliminating or reducing the incidence or occurrence of reactivation of latent HV infection.

The phrase “therapeutically effective amount” means an amount of a compound of the present invention that (i) treats the viral infection, (ii) attenuates, ameliorates, or eliminates one or more symptoms of the viral infection, or (iii) prevents or delays the onset of one or more symptoms of the viral infection described herein. Furthermore, “therapeutically effective amount” is an amount of a drug that is low enough to be non-toxic, yet sufficient to achieve a therapeutic result, including eliminating, reducing, and/or slowing the progression of a condition or symptom thereof. The therapeutically effective amount may depend on biological factors. Achieving a therapeutic result can be measured by physician or other qualified medical personnel using objective evaluations known in the art, or it can be measured by individual, subjective patient assessment.

The phrase “pharmaceutically acceptable salt” as used herein, refers to pharmaceutically acceptable organic or inorganic salts of a compound of this disclosure. Exemplary salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate salts. A pharmaceutically acceptable salt may involve the inclusion of another molecule such as an acetate ion, a succinate ion or other counter ion. The counter ion may be any organic or inorganic moiety that stabilizes the charge on the parent compound, such as meglumine. Furthermore, a pharmaceutically acceptable salt may have more than one charged atom in its structure, such as dimeglumine. Instances where multiple charged atoms are part of the pharmaceutically acceptable salt can have multiple counter ions. Hence, a pharmaceutically acceptable salt can have one or more charged atoms and/or one or more counter ion.

The desired pharmaceutically acceptable salt may be prepared by any suitable method available in the art. For example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. Acids which are generally considered suitable for the formation of pharmaceutically useful or acceptable salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1 19; P. Gould, International J. of Pharmaceutics (1986) 33 201 217; Anderson et al, The Practice of Medicinal Chemistry (1996), Academic Press, New York; Remington's Pharmaceutical Sciences, 18.sup. th ed., (1995) Mack Publishing Co., Easton Pa.; and in The Orange Book (Food & Drug Administration, Washington, D.C. on their website). These disclosures are incorporated herein by reference thereto.

The phrase “pharmaceutically acceptable” indicates that the substance or composition must be compatible chemically and/or toxicologically, with the other ingredients comprising a formulation, and/or the mammal being treated therewith.

The terms “synergistic” and “synergy” as used herein refer to a therapeutic combination which is more effective than the additive effects of the two or more single agents (i.e., a super additive effect). A determination of a synergistic interaction between a NK-1R antagonist, or a pharmaceutically acceptable salt thereof, and one or more nucleoside analogs, or a pharmaceutically acceptable salt thereof, may be based on the results obtained from the assays described herein. The combinations provided by this disclosure have been evaluated in several mathematical models used to quantify synergism, additivism, and antagonism. The combination therapy may provide “synergy” and prove “synergistic”, i.e., the effect achieved when the active ingredients used together is greater than the sum of the effects that results from using the compounds separately. A synergistic effect may be attained when the active ingredients are administered: sequentially, concomitantly, or concurently. When delivered in alternation therapy, a synergistic effect may be attained when the compounds are administered or delivered sequentially, e.g., by different injections in separate syringes or in separate pills or tablets. In general, during alternation therapy, an effective dosage of each active ingredient is administered sequentially, i.e., serially, whereas in combination therapy, effective dosages of two or more active ingredients are administered together. Combination effects may be evaluated using the BLISS independence model. Bliss scores were calculated as described previously. (Borisy, A., et al., “Systematic discovery of multicomponent therapeutics” Proceedings of the National Academy of Sciences of the United States of America, 2003; 100(13):7977-82, Epub 2003 Jun. 12). BLISS scores quantify degree of potentiation from single agents and a BLISS score>0 suggests greater than simple additivity. Synergy scores were also computed using the Zero Interaction Potency (ZIP) model (Yadav, B. et al., “Searching for Drug Synergy in Complex Dose-Response Landscapes Using an Interaction Potency Model.” Computational and Structural Biotechnology Journal, 13:504-13. 25 Sep. 2015).

The term “subject” refers to animals such as mammals, including, but not limited to, primates (e.g., humans), cattle, poultry/avian, sheep, horses, goats, dogs, cats, rabbits, rats, mice and the like. In the methods of this disclosure, the subject is typically a human. At risk subjects appropriate for treatment include subjects exposed to other subjects having a herpesviridae infection or reactivation (infectious or contagious), or where the risk of herpesviridae infection is increased due to changes in virus infectivity or cell tropism, immunological susceptibility (e.g., an immunocompromised subject), or environmental risk. At risk subjects appropriate for treatment therefore include human subjects exposed to, or at risk of, exposure to other humans that have herpesviridae infection or reactivation (infectious or contagious) or are at risk of a herpesviridae infection or reactivation (infectious or contagious). Subjects further include immunocompromised subjects due to an immunological disorder (e.g., autoimmunity) or disease, or an immune-suppressing treatment (e.g., cyclophosphamide). Subjects also include those having been exposed to or diagnosed as HIV positive. Subjects further include those receiving or candidates for a tissue or organ transplant.

The term “prophylaxis” or “prophylactic” refers to the continued absence of symptoms of the infection or related pathology, disease, or condition that would be expected had the combination of NK-1R antagonist and nucleoside analog not been administered. In the context of HV infection, prophylaxis specifically includes the treatment of naïve subjects (i.e., subjects who are not infected with HV) who may be exposed HV and may be at higher risk of infection. Non-limiting examples include newborns born to a mother infected with HV, immunocompromised children or adults, and unvaccinated parents of school-aged children who may transmit VZV to family members.

As used herein, a “kit” is a commercial unit of sale, which may comprise a fixed number of doses of the one or more pharmaceutical compositions. By way of example only, a kit may provide a 30-day supply of dosage units of one or more fixed strengths, the kit comprising 30 dosage units, 60 dosage units, 90 dosage units, 120 dosage units, or other appropriate number according to a health care provider's instruction. As another example, a kit may provide a 90-day supply of dosage units.

Additional definitions are provided below as appropriate.

### Blocking Neurokinin-1 Receptor Signaling

Agents that block or inhibit NK-1 signaling include but are not limited to peptides, antibodies, antisense oligonucleotides, morpholinos, or small molecule inhibitors, such as receptor antagonists. The agent that inhibits CD47 signaling includes, in various embodiments, a synthetic peptide having specific binding affinity for the NK-1 receptor (NK- 1R); an oligonucleotide comprising at least about 15 contiguous bases and that hybridizes to the mature or unprocessed nuclear mRNA of NK-1R under high stringency conditions; an isolated or recombinant NK-1R molecule or soluble fragment thereof, or molecule that binds thereto; an agent that decreases the expression of NK-1R; an agent that enhances the proteolysis of NK-1R; an agent that enhances removal of NK-1R from the cell surface; a NK-1R antagonist; an antibody that specifically binds NK-1R; or a mixture of two or more thereof.

NK-1 receptor antagonists are a class of antiemetic drugs that possess antiemetic properties. The discovery of NK-1 receptor antagonists was a turning point in the prevention of emesis associated with cancer chemotherapy as they can prevent both central and peripheral stimulation of vomiting centers. NK-1 receptor antagonists have recently been shown to decrease or inhibit herpesviridae infection or pathogenesis, or a symptom or pathology associated with a herpesviridae infection or pathogenesis, or an adverse side effect of herpesviridae infection or pathogenesis, using the current inventors' in vitro model, as described in PCT Application No. PCT/US2019/024521 (published as WO 2019/191391), which is incorporated herein in its entirety. This model demonstrates a novel pro-viral function of nuclear NK-1R in the absence of its endogenous ligand, substance P, that results in formation of lamellipodia and filopodia, which provide “highways” for cell-to-cell spread of VZV. The description of VZV-exploited NK-1R function in this study was critical to the knowledge of VZV pathogenesis in the central nervous system. Targeting NK-1R with an antagonist provides an antiviral therapy against VZV and a much-needed alternative/adjuvant to treatment of recurrent or disseminated VZV infections.

The NK-1 receptor is a receptor that binds Substance P (an 11 amino acid polypeptide). An NK-1 receptor antagonist is a compound that directly or indirectly decreases the biological activity of an NK-1 receptor by inhibiting a signaling pathway regulated by Substance P binding to the receptor. The NK-1 receptor biological activity is reduced by 10%, 20%, 30%, 40%, 50%, 70%, 80%, 90%, or even 100% relative to a control. Exemplary NK-1 receptor antagonists useful in the methods of this disclosure include aprepitant, rolapitant, casopitant, fosaprepitant, netupitant, and maropitant, each of which are effective to treat postsurgical nausea and vomiting and cancer chemotherapy-induced nausea and vomiting. Aprepitant is a highly selective NK-1 receptor antagonist which crosses the blood-brain barrier and occupies NK-1 receptors in the brain. Aprepitant is available commercially (Emend® and Cinvanti®) for oral or intravenous (IV) administration, fosaprepitant (Emend® for Injection) is available commercially as an IV formulation.

### Nucleoside Analogs

Nucleoside analogues are nucleosides that contain a nucleic acid analogue and a sugar. The most commonly used nucleoside analog is acyclovir, although it contains no actual sugar, as the sugar ring is replaced by an open-chain structure. The nucleoside analogues are an important class of antiviral agents now commonly used in the therapy of herpes simplex virus (HSV), varicella-zoster (VZV) infection, human immunodeficiency virus (HIV) infection, hepatitis B virus (HBV), hepatitis C virus (HCV), and cytomegalovirus (CMV). The nucleoside analogues resemble naturally occurring nucleosides and act by causing termination of the nascent DNA chain or by competitive inhibition of the viral polymerase. These agents are generally safe and well tolerated as they are used by the viral, but not human polymerases in DNA replication. Exemplary NK-1 receptor antagonists useful in the methods of this disclosure include acyclovir (Sitavig®, Zovirax®), valacyclovir (Valtrex®), cidofovir (Vistide®) penciclovir (Denavir®), famciclovir (Famvir®), ganciclovir (Cytovene®), valganciclovir (Valcyte®), idoxuridine (Herplex®, Dendrid®), trifluridine (Viroptic®), and vidarabine (Vira-A®).

### Pharmaceutical Compositions and Administration

In the methods of treating infection or reactivation of herpesviridae provided by this disclosure, the synergistic combination of a NK-1R antagonist, such as aprepitant, and a nucleoside analog, such as acyclovir, may be administered to a subject sequentially (e.g. by different injections or in separate capsules or tablets), concomitantly (e.g., separately but within about 30 minutes or less of one another), or concurrently (e.g., administered to the subject within a time sufficiently close such that each of these agents remains at or attains a therapeutically effective drug level within the subject such that the synergistic effect of the combination is experienced by the subject).

The NK-1R antagonist and the nucleoside analog may also be administered as a combined formulation. Typically, a pharmaceutical composition includes a pharmaceutically acceptable carrier such as solvents, dispersion media, coatings, antibacterial and antifungal agents, isotonic and absorption delaying agents, and the like that are physiologically compatible. The composition can include a pharmaceutically acceptable salt. Pharmaceutical formulation is a well-established art, and is further described, e.g., in Gennaro (ed.), Remington: The Science and Practice of Pharmacy, 20th ed., Lippincott, Williams & Wilkins (2000)); Ansel et al., Pharmaceutical Dosage Forms and Drug Delivery Systems, 7.sup.th Ed., Lippincott Williams & Wilkins Publishers (1999) (ISBN: 0683305727); and Kibbe (ed.), Handbook of Pharmaceutical Excipients American Pharmaceutical Association, 3rd ed. (2000).

The synergistic combination of a NK-1R antagonist, such as aprepitant, and a nucleoside analog, such as acyclovir can be administered to a subject, e.g., a subject in need thereof, by a variety of methods, including for example, intravenous injection or infusion (IV), subcutaneous injection (SC), intraperitoneally (IP), or intramuscular (IM) injection, orally, topically, vaginally, or rectally. Other modes of parenteral administration may rarely be used, including, for example, intraarterial, intrathecal, intracapsular, intraorbital, intracardiac, intradermal, transtracheal, subcuticular, intraarticular, subcapsular, subarachnoid, intraspinal, epidural, and intrasternal injection.

The route and dosage of administration of the synergistic combination of a NK-1R antagonist and nucleoside analog can be tailored for the individual case, e.g., by diagnosing and/or monitoring the subject and administering the combination as a fixed dose, or in a mg/kg patient weight dose of each drug. Dosage regimens are adjusted to provide the desired response, e.g., a therapeutic response or a combinatorial synergistic therapeutic effect. Factors that may influence the dosage and timing required to effectively treat a subject, include, e.g., the severity of the disease or disorder, formulation, route of delivery, previous treatments, the general health and/or age of the subject, and other diseases present. Moreover, treatment of a subject with a therapeutically effective amount of a compound can include a single treatment or, preferably, can include a series of treatments.

Methods of this disclosure are applicable to providing a subject with protection against herpesviridae infection, reactivation, or pathogenesis, treating a subject for herpesviridae infection, reactivation and pathogenesis; and decreasing susceptibility or inhibiting herpesviridae reactivation from latency in a subject. The methods of this disclosure are therefore applicable to herpesviridae infection that is in an active state, latent state, or reactivated state.

The term “infection,” when used in reference to herpesviridae, means an initial or primary infection. An infection may be “infectious” in the sense that herpesviridae infects other sites in the infected host subject, or contagious to other subjects (cross-infection), or may be latent, in which case herpesviridae does not generally infect other sites or is contagious to other subjects. In immunocompetent subjects, initial/primary infection is usually either asymptomatic or causes mild pathogenesis or symptoms; only a small proportion of subjects develop more severe clinical illness. Primary infection is self-limiting in immunocompetent patients. In contrast, primary herpesviridae infection in immunocompromised subjects (e.g., immunosuppressant treatment, HIV+, newborns/neonates, pregnant, elderly subjects, etc.), can result in severe symptoms and may even be fatal.

Following a primary or initial herpesviridae infection, the virus establishes “latency,” in the host subject which allows the virus to evade immune clearance and remain in the host subject, and infection is lifelong. In the latent state, herpesviridae does not typically cause illness or symptoms, there is little if any viral replication and the subject is not infectious or contagious. Latency, also referred to as “latent infection” may occur in a different cell type from that of the initial/primary herpesviridae infection.

Reactivation, when used in reference to herpesviridae, means activation of herpesviridae in the host subject following a period of latency. Reactivation is associated with increased viral replication and proliferation in an herpesviridae infected host subject, who becomes infectious and contagious again. Symptoms and pathologies associated with or caused by herpesviridae reactivation may or may not be the same type, severity, frequency, or duration as initial herpesviridae infection and subsequent pathogenesis. For example, VZV/HHV-3 causes chickenpox (primary infection) and shingles (reactivation). Reactivation can be milder (e.g., asymptomatic) than an initial herpesviridae infection/pathogenesis, in which case it would not be obvious whether a host subject is in a latent or reactivated state. In immunocompetent host subjects, reactivation is typically mild, whereas in immunocompromised host subjects, symptoms associated with or caused by reactivation can be severe and lead to death. Thus, clinical manifestations associated with reactivation may be different from that observed with an initial/primary infection. Accordingly, a single herpesviridae infection can cause different clinical symptoms or pathologies. One symptom of herpesviridae reactivation is the appearance of “cold sores” around mucosal areas (e.g., mouth, lips, tongue, genitalia, etc.). Reactivation occurs periodically and can be induced by stress, immune suppression, etc.

Specific examples of symptoms and pathologies associated with or caused by herpesviridae infection, reactivation or pathogenesis, whose onset, progression, severity, frequency, duration, or probability can be reduced, decreased, inhibited, delayed, ameliorated or prevented include, for example, lesions, ulcers, canker sore, cold sore, rash, boils, Gingivostomatitis, Herpetic whitlow Traumatic herpes (herpes gladiatorum), Eczema herpeticum, fever, fatigue, headache, sore throat, swollen lymph nodes, pneumonitis, pneumonia, hepatitis, meningitis, myelitis, myelopathy, neuropathy, pancreatitis, VZV-associated diabetic complications, vasculopathy, Encephalitis, keratitis, Genital herpes, esophagitis, dysphasia, hemiparesis, coma, shingles, chicken pox, mononucleosis, chronic or acute pelvic inflammatory disease (PID), proctitis, colitis, nerve damage and death. Other symptoms and pathologies of herpesviridae infection, reactivation or pathogenesis, are known in the art and treatment thereof in accordance with the methods of this disclosure is provided.

The methods of this disclosure, including, among other methods, providing a subject with protection against a herpesviridae infection, reactivation or pathogenesis, treatment of a herpesviridae infection, reactivation or pathogenesis, or a symptom or pathology associated with or caused by herpesviridae infection, reactivation or pathogenesis, or decreasing susceptibility of a subject to a herpesviridae infection, reactivation or pathogenesis, can therefore result in an improvement in the subjects' condition. An improvement is therefore any objective or subjective reduction, decrease, inhibition, delay, ameliorating, or prevention of onset, progression, severity, duration, frequency or probability of one or more symptoms or pathologies associated with or caused by herpesviridae infection, reactivation or pathogenesis (e.g., illness), or virus titer, viral load, replication, proliferation, or an amount of a viral protein. An improvement would also include reducing, inhibiting, or preventing increases in virus titer, viral load, replication, proliferation, or an amount of a viral protein of one or more herpesviridae strains or isolates or subtypes or species. An improvement would further include stabilizing a symptom or pathology associated with or caused by herpesviridae infection, reactivation, or pathogenesis, or inhibiting, decreasing, delaying, or preventing a worsening or progression of the symptom or pathology associated with or caused by herpesviridae infection, reactivation, or pathogenesis, or progression of the underlying herpesviridae infection. An improvement can therefore be, for example, in any of lesions, ulcers, canker sore, cold sore, rash, boils, Gingivostomatitis, Herpetic whitlow Traumatic herpes (herpes gladiatorum), Eczema herpeticum, fever, fatigue, headache, sore throat, swollen lymph nodes, pneumonitis, pneumonia, hepatitis, meningitis, myelitis, myelopathy, neuropathy, pancreatitis, VZV-associated diabetic complications, vasculopathy, Encephalitis, keratitis, genital herpes, esophagitis, dysphasia, hemiparesis, coma, shingles, chicken pox, mononucleosis, chronic or acute pelvic inflammatory disease (PID), proctitis, colitis, nerve damage, and death, to any degree or for any duration of time (hours, days, weeks, months, years, or cure).

An improvement would also include reducing or eliminating a need, dosage amount, or frequency of another treatment, such as an antiviral drug or other agent used for treating a subject having, or at risk of having, a herpesviridae infection, reactivation, or pathogenesis, a symptom or pathology associated with or caused by herpesviridae infection, reactivation or pathogenesis, or decreasing or preventing an adverse side effect caused by vaccination with or against a herpesviridae. Thus, reducing an amount of another treatment for herpesviridae infection, reactivation or pathogenesis, a symptom or pathology associated with or caused by herpesviridae, or an adverse side effect caused by vaccination with or against a herpesviridae is considered to provide a benefit and, therefore, is considered within the methods of this disclosure. Non-limiting exemplary herpesviridae treatments that may be eliminated or used at reduced doses or frequencies of administration include protease inhibitors, reverse transcriptase inhibitors, virus fusion inhibitors, and virus entry inhibitors.

A treatment or improvement need not be complete ablation of any particular infection, reactivation, pathogenesis, symptom, pathology or adverse side effect, or all of the infection, reactivation, pathology, symptoms, pathologies or adverse side effects associated with or caused by herpesviridae infection, reactivation or pathogenesis, or vaccination with or against herpesviridae. Rather, treatment may be any objective or subjective measurable or detectable anti-virus effect or improvement in a treated subject. Thus, reducing, inhibiting, decreasing, eliminating, delaying, halting, or preventing a progression or worsening of the infection, reactivation or pathogenesis, a symptom or pathology of the infection, or an adverse side effect caused by vaccination is a satisfactory outcome. For example, an NK-1R antagonist may reduce, inhibit, delay formation of, or stabilize lesions, ulcers, canker sores, or cold sores, but not have a measurable effect on rash, boils, Gingivostomatitis, Herpetic whitlow Traumatic herpes (herpes gladiatorum), Eczema herpeticum, fever, fatigue, headache, sore throat, swollen lymph nodes, pneumonitis, pneumonia, hepatitis, meningitis, myelitis, myelopathy, neuropathy, pancreatitis, VZV-associated diabetic complications, vasculopathy, Encephalitis, keratitis, genital herpes, esophagitis, dysphasia, hemiparesis, coma, shingles, chicken pox, mononucleosis, chronic or acute pelvic inflammatory disease (PID), proctitis, colitis, nerve damage or death. Another example is where an NK-1R antagonist reduces fever or fatigue, without a detectable improvement in one or more other symptoms or pathologies. Thus, a satisfactory clinical endpoint is achieved when there is an incremental improvement in the subject's condition or a partial reduction or a stabilization of a herpesviridae infection, reactivation, pathogenesis or a symptom, pathology or adverse side effect thereof, or an inhibition or prevention of worsening or progression of the herpesviridae infection, reactivation, pathogenesis, symptom, pathology or adverse side effect thereof (stabilizing one or more symptoms or pathologies), over a short or long duration (hours, days, weeks, months, years, or cure).

In the methods of this disclosure in which there is a desired outcome, for example, a therapeutic or prophylactic method that provides an objective or subjective improvement in a herpesviridae infection, reactivation or pathogenesis, a symptom or pathology associated with or caused by herpesviridae, or an adverse side effect caused by vaccination with or against herpesviridae or an herpesviridae treatment, an NK-1R antagonist may be administered in a sufficient or effective amount. As used herein, a “sufficient amount” or “effective amount” or an “amount sufficient” or an “amount effective” refers to an amount that provides, in single or multiple doses, alone or in combination with one or more other compounds, treatments, agents (e.g., a drug) or therapeutic regimens, a long term or a short term detectable or measurable improvement or beneficial effect to a given subject of any degree or for any time period or duration (e.g., for minutes, hours, days, months, years, or cured). A “sufficient amount” or “effective amount” therefore includes an amount sufficient to result in decreasing, reducing, inhibiting, preventing, or delaying onset; decreasing, reducing, inhibiting, delaying, or preventing a progression or worsening of; or reducing, relieving, ameliorating, or alleviating, severity, frequency, duration, susceptibility, or probability of herpesviridae infection, reactivation or pathogenesis, one or more symptoms associated with or caused by herpesviridae infection, reactivation or pathogenesis, or an adverse side effect of vaccination with or against a herpesviridae or an herpesviridae treatment. In addition, hastening a subject's recovery from herpesviridae infection, reactivation or pathogenesis, one or more symptoms associated with or caused by herpesviridae infection, reactivation or pathogenesis, or an adverse side effect of vaccination with or against a herpesviridae or an herpesviridae treatment is considered to be a sufficient or effective amount. Various beneficial effects and indicia of therapeutic and prophylactic benefit are set forth herein and are known to the skilled artisan.

A sufficient amount or an effective amount can but need not be provided in a single administration and can but need not be administered alone (i.e., without a third drug, agent, treatment or therapeutic regimen), or in combination with another compound, agent, treatment or therapeutic regimen. In addition, a sufficient amount or an effective amount need not be sufficient or effective if given in single or multiple doses without a second compound, treatment, agent, or therapeutic regimen, because additional doses, amounts, frequency or duration of administration above and beyond such doses, or additional compounds, agents, treatments or therapeutic regimens may be included in order to be effective or sufficient in a given subject. A sufficient amount or an effective amount need not be effective in each and every subject, nor a majority of subjects in a given group or population. Thus, a sufficient amount or an effective amount means sufficiency or effectiveness in a particular subject, not a group or the general population. As is typical for such methods, some subjects will exhibit a greater or lesser response to a method of this disclosure than other subjects. Amounts, frequencies, or duration also considered sufficient and effective and are therefore beneficial are those that result in the elimination or a reduction in amount, frequency or duration of another compound, agent, treatment or therapeutic regimen. For example, an NK-1R antagonist is considered as having a beneficial or therapeutic effect if contact, administration, or delivery in vivo results in the use of a lesser amount, frequency or duration of a nucleoside analog.

The synergistic combination is for providing a subject with protection against herpesviridae infection, reactivation or pathogenesis; decreasing susceptibility of a subject to a herpesviridae infection, reactivation or pathogenesis; or decreasing or preventing an adverse side effect caused by herpesviridae vaccination or an herpesviridae treatment.

### Kits Comprising Pharmaceutical Compositions and a Package Insert

An aspect of this disclosure provides kits containing one or more NK-1R antagonists and one or more nucleoside analogs, either in the same pharmaceutical composition or different pharmaceutical compositions, and a package insert.

In exemplary embodiments, the kit may comprise a pharmaceutical composition comprising one or more NK-1R antagonists and one or more nucleoside analogs according to the present disclosure, and a package insert. Similarly, a kit may comprise a pharmaceutical composition comprising one or more NK-1R antagonists and a separate pharmaceutical composition comprising one or more nucleoside analogs according to the present disclosure, and a package insert. As used herein, “package insert” means a document which provides information on the use of the one or more pharmaceutical compositions, safety information, and other information required by a regulatory agency. A package insert can be a physical printed document in some embodiments. Alternatively, a package insert can be made available electronically to the user. The package insert may inform a user of the kit that the pharmaceutical composition(s) may be administered according to the methods of use of the present invention.

An example of such a kit in relation to solid dosage forms is a so-called blister pack. Blister packs are well known in the packaging industry and are being widely used for the packaging of pharmaceutical unit dosage forms (tablets, capsules, and the like).

Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil that is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening. As noted above, the kit may include instructions for coordinating the administration of the NK-1R antagonist and the nucleoside analog as part of an HV treatment or prevention regimen disclosed herein.

Advantageously, the blister pack may be marked or colored in a manner that may increase compliance with the dosing regimen of the NK-1R antagonist and the nucleoside analog.

The invention will be further illustrated with reference to the following non-limiting Figures and Examples. It will be apparent to those skilled in the art that many changes can be made in the embodiments described without departing from the scope of the present invention. Thus, the scope of the present invention should not be limited to the embodiments described in this application, but only by embodiments described by the language of the claims and the equivalents of those embodiments.

### EXAMPLE

### Synergistic Effect of Aprepitant and Acyclovir

Primary human spinal astrocytes (HA-sp; Sciencell, Carlsbad, Calif.) were seeded at 5,000 cells/cm² in a basal astrocyte medium containing 2% fetal bovine serum (FBS), 1% astrocyte growth supplement, and 1% 100× penicillin-streptomycin (Sciencell). After 24 hours, medium was changed to basal astrocyte medium containing 0.1% FBS and 1% 100× penicillin-streptomycin that was replenished every 72 hours for 7 days to establish quiescence. On day 7, spinal astrocytes were co-cultivated with VZV-infected spinal astrocytes (40 pfu/mL) or mock-infected spinal astrocytes. Aprepitant and acyclovir application: HA-sp were VZV-infected as described above and incubated for 12 hours to establish productive infection. At 12 hours post-infection (HPI), VZV-infected spinal astrocytes were treated with 10 μM aprepitant (the optimal concentration to avoid cell death was determined in a preliminary toxicity assay on uninfected HA-sp using aprepitant at 1-100 μM; data not shown) and/or acyclovir 1-100 μM or vehicle (DMSO) and treated again at 24 HPI and 48 HPI. Light microscopy images to observe a cytopathic effect were obtained at 12, 24, 48 and 72 HPI followed by DNA extraction and PCR quantification of VZV DNA. Mock-and VZV-infected HA-sp were harvested and placed into 200 μL of lysis buffer with proteinase K and incubated for 20 minutes at 56° C. (DNeasy Blood and Tissue Kit; Qiagen, Germantown, Md.). DNA was extracted per the manufacturer's instructions and eluted in 100 μL of nuclease-free water. DNA was then analyzed by quantitative PCR using primers corresponding to sequences in VZV ORF 68 and in cellular glyceraldehyde-3-phosphate-dehydrogenase (GAPdH) as previously described (Cohrs and Gilden, J Virol 2007; 81: 2950-6). Data were normalized to GAPDH and analyzed using the delta delta threshold cycle (C_{T}) method. Statistical Analysis: Statistical analysis was performed using GraphPad Prism (GraphPad, San Diego, Calif.). Significance of differences in VZV DNA between treated and untreated VZV-infected HA-sp at each time point was determined using the Student's paired t test.

Primary human spinal astrocytes were infected with VZV and treated 18 hours later with various doses of aprepitant, acyclovir, or a combination of the two drugs.

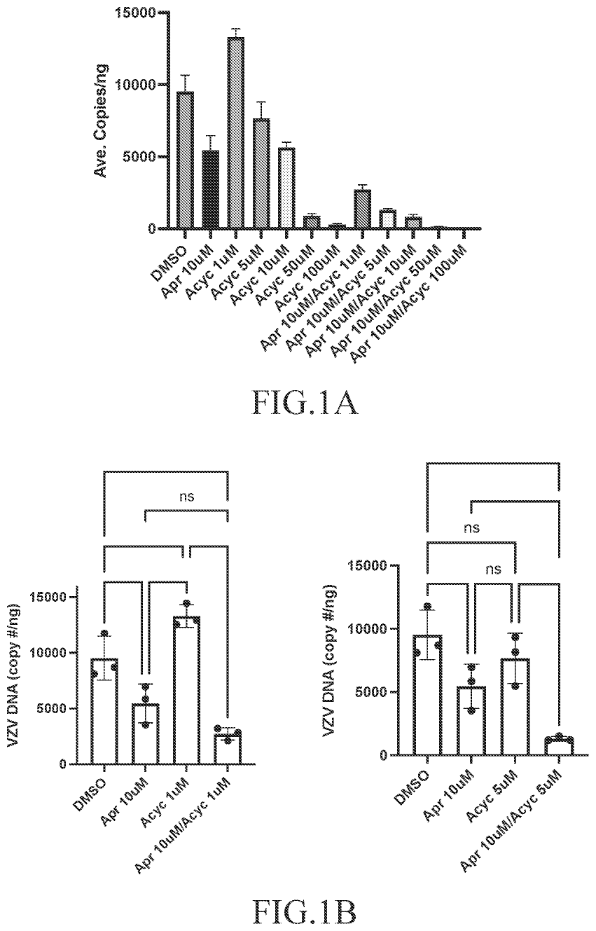

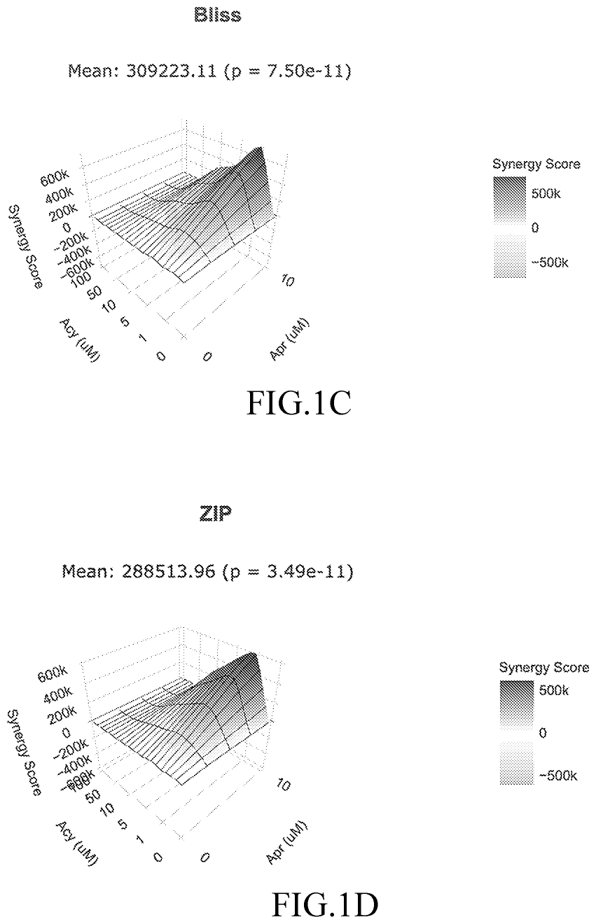

Twelve separate treatment groups were tested, as follows:
1. DMSO control;
2. Aprepitant (Apr) 10 μM;
3. Acyclovir (Acyc) 1 μM, 5 μM, 10 μM, 50 μM, and 100 μM;
4. Aprepitant 10 μM combined with acyclovir 1 μM, 5 μM, 10 μM, 50 μM, and 100 μM.
   The cells were harvested 48 hours later and DNA was extracted. VZV DNA was quantified by RT-qPCR. FIG. 1A shows the average VZV DNA copy numbers in all treatment groups. FIG. 1B shows individual statistical comparisons for select treatment combinations. FIG. 1C shows BLISS mathematical modeling for drug synergy, showing a significant positive synergistic (super additive) effect at reducing VZV DNA copy number when both drugs were combined. FIG. 1D shows ZIP mathematical modeling for drug synergy, showing a significant positive synergistic (super additive) effect at reducing VZV DNA copy number when both drugs were combined. The most robust response was seen with aprepitant 10 μM dosed with acyclovir at either 1 μM or 5 μM.

The various features and processes described above may be used independently of one another, or may be combined in various ways. All possible combinations and subcombinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

What is claimed is:

1. A method of treating or preventing infection or reactivation of herpesviridae in a subject comprising administering to the subject a synergistic combination of:
(i) an agent that blocks Neurokinin-1 Receptor (NK-1R) signaling; and,
(ii) a nucleoside analog selected from the group consisting of acyclovir, famciclovir, valaciclovir, and pharmaceutically acceptable salts thereof.

2. The method of claim 1, wherein the agent that blocks NK-1R signaling is an NK-1R antagonist, an anti-NK-1R antibody or fragment thereof, a NK-1R-binding peptide, a NK-1R antisense oligonucleotide, or a NK-1R morpholino.

3. The method of claim 1 or 2, wherein the agent that blocks NK-1R signaling is an NK-1R antagonist selected from the group consisting of aprepitiant, rolapitant, fosaprepitant, and pharmaceutically acceptable salts thereof.

4. The method of claim 2, wherein the NK-1R antagonist and the nucleoside analog are administered sequentially.

5. The method of claim 2, wherein the NK-1R antagonist and the nucleoside analog are administered concomitantly.

6. The method of claim 2, wherein the NK-1R antagonist and the nucleoside analog are administered concurrently.

7. The method of claim 2, wherein the NK-1R antagonist and the nucleoside analog are administered as a combined formulation.

8. The method of any one of claims 1-7, wherein the NK-1R antagonist is aprepitant.

9. The method of any one of claims 1-7, wherein the NK-1R antagonist is rolapitant.

10. The method of any one of claims 1-7, wherein the NK-1R antagonist is fosaprepitant.

11. The method of any one of claims 1-10, wherein the nucleoside analog is acyclovir.

12. The method of any one of claims 1-10, wherein the nucleoside analog is famciclovir.

13. The method of any one of claims 1-10, wherein the nucleoside analog is valaciclovir.

14. The method of any one of claims 1-14, wherein the NK-1R antagonist is administered orally.

15. The method of any one of claims 1-14, wherein the NK-1R antagonist is administered intravenously.

16. The method of any one of claims 1-14, wherein the nucleoside analog is administered orally.

17. The method of any one of claims 1-14, wherein the nucleoside analog is administered intravenously.

18. The method of any one of claims 1-14, wherein the nucleoside analog is administered topically.

19. The method of any one of claims 1-18, wherein the herpesviridae is an alpha-herpesvirus.

20. The method of any one of claims 1-18, wherein the herpesviridae is a beta-herpesvirus.

21. The method of any one of claims 1-18, wherein the herpesviridae is a gamma-herpesvirus.

22. The method of any one of claims 1-18, wherein the herpesviridae is herpes simplex type 1 (HSV-1).

23. The method of any one of claims 1-18, wherein the herpesviridae is herpes simplex type 2 (HSV-2).

24. The method of any one of claims 1-18, wherein the herpesviridae is varicella-zoster virus (VZV).

25. The method of any one of claims 1-18, wherein the herpesviridae is Epstein-Bar virus (EBV).

26. The method of any one of claims 1-18, wherein the herpesviridae is Kaposi's sarcoma-associated herpesvirus (KSHV).

27. The method of any one of claims 1-26, wherein the herpesviridae in the subject is in a latent state

28. The method of any one of claims 1-26, wherein the herpesviridae in the subject is in an active state.

29. The method of any one of claims 1-26, wherein the herpesviridae in the subject is in a reactivated state.

30. The method of any one of claims 1-29, wherein the administration of the synergistic combination reduces, decreases, inhibits, or ameliorates the onset, severity, duration, progression, or frequency of one or more symptoms or pathologies associated with or caused by herpesviridae infection or pathogenesis, or reactivation of herpesviridae from latency, in the subject.

31. The method of claim 30, wherein the symptoms or pathologies treated include myelitis, myelopathy, neuropathy, pancreatitis, VZV-associated diabetic complications, vasculopathy, lesions, ulcers, canker sore, close sore, rash, boils, gingivostomatitis, herpes gladiatorum, eczema herpeticum, swollen lymph nodes, pneumonitis, pneumonia, hepatitis, meningitis, encephalitis, keratitis, genital herpes, esophagitis, hemiparesis, shingles, chicken pox, mononucleosis, chronic or acute pelvic inflammatory disease (PID), proctitis, colitis, and/or nerve damage.

32. The method of claim 30, wherein the herpesviridae infection is in spinal astrocytes in the subject and the pathogenesis is VZV myelitis and/or VZV myelopathy.

33. The method of claim 30, wherein the herpesviridae infection is in perineurial cells in the subject and the pathogenesis is VZV neuropathy.

34. The method of claim 30, wherein the herpesviridae infection is in pancreatic islet cells in the subject and the pathogenesis is VZV pancreatitis and/or VZV-associated diabetic complications.

35. The method of claim 30, wherein the herpesviridae infection is in keratocytes in the subject and the pathogenesis is VZV keratitis.

36. The method of claim 30, wherein the herpesviridae infection is in brain vascular adventitial fibroblasts in the subject and the pathogenesis is VZV vasculopathy.

37. The method of any one of claims 1-36, wherein the administration of the synergistic combination stabilizes the herpesviridae infection or one or more symptoms or pathologies associated with the herpesviridae infection or pathogenesis or reactivation of herpesviridae from latency.

38. The method of any one of claims 1-36, wherein the administration of the synergistic combination reduces herpesviridae titer, viral load, viral replication, viral proliferation or viral protein production, or inhibits increases in herpesviridae titer, viral load, viral replication, viral proliferation, or viral protein production.

39. The method of any one of claims 1-36, wherein the subject is immunocompromised, or is a candidate for or has received an immunosuppressant treatment, or is a candidate for or has received a tissue or organ transplant.

40. A kit comprising:
(i) a pharmaceutical composition comprising a Neurokinin-1 Receptor (NK-1R) antagonist selected from the group consisting of aprepitant, rolapitant, fosaprepitant, and pharmaceutically acceptable salts thereof; and,
(ii) a pharmaceutical composition comprising a nucleoside analog selected from the group consisting of acyclovir, famciclovir, valaciclovir, and pharmaceutically acceptable salts thereof; and,
(iii) a package insert.

41. A kit comprising
a pharmaceutical composition comprising:
(i) a Neurokinin-1 Receptor (NK-1R) antagonist selected from the group consisting of aprepitant, rolapitant, fosaprepitant, and pharmaceutically acceptable salts thereof; and,
(ii) a nucleoside analog selected from the group consisting of acyclovir, famciclovir, valaciclovir, and pharmaceutically acceptable salts thereof; and,
a package insert.

42. The kit of claim 40 or 41, wherein the NK-1R antagonist and the nucleoside analog are provided in the form of a blister pack.
